SEQUENCE LISTING (24 sequences)

SEQ ID NO: 1 - protein, 16 aa, Drosophila sp.
RQIKIWFQNRRMKWKK

SEQ ID NO: 2 - protein, 13 aa, Human immunodeficiency virus
GRKKRRQRRRPPQ

SEQ ID NO: 3 - protein, 15 aa, Homo sapiens
AAVLLPAVLLALLAP

SEQ ID NO: 4 - protein, 16 aa, Artificial Sequence
  Arg/Trp Analogue
RRWRRWWRRWWRRWRR

SEQ ID NO: 5 - protein, 9 aa, Artificial Sequence
  Oligomeric D-arginine
RRRRRRRRR

SEQ ID NO: 6 - protein, 34 aa, human herpesvirus 1
DAATATRGRSAASRPTERPRAPARSASAPAAPVG

SEQ ID NO: 7 - protein, 13 aa, Artificial Sequence
  D-Tat
GRKKRRQRRRPPQ

SEQ ID NO: 8 - protein, 13 aa, Artificial Sequence
  R9-Tat
GRRRRRRRRRPPQ

SEQ ID NO: 9 - protein, 12 aa, Artificial Sequence
  U2AF (142-153)
SQMTRQARRLYV

SEQ ID NO: 10 - protein, 18 aa, Human immunodeficiency virus type 1
TRQARRNRRRRWRERRQR

SEQ ID NO: 11 - protein, 15 aa, flock house virus
RRRRNRTRRNRRRVR

SEQ ID NO: 12 - protein, 19 aa, Brome mosaic virus
KMTRAQRRAAARKNKRNTR

SEQ ID NO: 13 - protein, 13 aa, Human lymphotropic virus type III
TRRQRTRRARRNR

SEQ ID NO: 14 - protein, 19 aa, Cowpea chlorotic mottle virus
KLTRAQRRAAARKNKRNTR

SEQ ID NO: 15 - protein, 17 aa, Artificial Sequence
  P22 N (14-30)
NAKTRRHERRRKLAIER

SEQ ID NO: 16 - protein, 27 aa, Artificial Sequence
  cFOS (139-164)
KRRIRRERNKMAAAKSRNRRRELTDDT

SEQ ID NO: 17 - protein, 28 aa, Artificial Sequence
  cJUN (252-279)
RIKAERKRMRNRIAASKSRKRKLERIAR

SEQ ID NO: 18 - protein, 22 aa, Artificial Sequence
  GCN4 (231-252)
KRARNTEAARRSRARKLQRMQK

SEQ ID NO: 19 - protein, 12 aa, Artificial Sequence
  PTD-4
PIRRRKKLRRLK

SEQ ID NO: 20 - protein, 12 aa, Artificial Sequence
  PTD-5
RRQRRTSKLMKR

SEQ ID NO: 21 - protein, 13 aa, Artificial Sequence
  Penetratin
GRKKRRQRRRPPQ

SEQ ID NO: 22 - protein, 26 aa, Artificial Sequence
  Transportan
GWTLNSAGYLLKINLKALAALAALIL

SEQ ID NO: 23 - protein, 18 aa, Artificial Sequence
  Amphipathic peptide
KLALKLALKALKAALKLA

SEQ ID NO: 24 - protein, 11 aa, Human immunodeficiency virus type 1
YGRKKRRQRRR

(19) United States
(12) Patent Application Publication
(10) Pub. No.: US 2005/0003546 A1
(43) Pub. Date: Jan. 6, 2005

(54) Viral uptake into cells and tissues

(75) Inventors: William C. Sessa, Madison, CT (US); Jean-Philippe Gratton, Montreal (CA)

Correspondence Address: MORGAN, LEWIS & BOCKIUS LLP, 1701 MARKET STREET, PHILADELPHIA, PA, 19103-2921, US

(73) Assignee: Yale University

(21) Appl. No.: 10/738,632

(22) Filed: Dec. 17, 2003

Related U.S. Application Data
(63) Continuation of application No. PCT/US02/20337, filed on Jun. 26, 2002.
(60) Provisional application No. 60/303,117, filed on Jul. 5, 2001.

(51) Int. Cl. C12N 5/08; C12N 15/867

(52) U.S. Cl. 435/456; 435/366

(57) ABSTRACT

The invention relates to compositions and methods for facilitating fusion of a virus with a cell and for facilitating virus-mediated transduction of a nucleic acid into a cell. The invention further relates to the use of cell permeable peptides to facilitate fusion of a virus with a cell.

### CROSS REFERENCE TO RELATED APPLICATIONS

The present application claims priority under 35 U.S.C. § 119(e) to U.S. Provisional Patent Application No. 60/303,117, filed Jul. 5, 2001.

### STATEMENT REGARDING FEDERALLY SPONSORED RESEARCH AND DEVELOPMENT

This invention was made in part using U.S. Government support (NIH Grants HL57665, HL61371, and HL64793) and the U.S. Government may therefore have certain rights in the invention.

### BACKGROUND OF THE INVENTION

Targeted gene delivery in humans has been limited by the efficiency of in vivo DNA transfer. The use of high titer viral vectors in order to achieve acceptable gene expression is frequently associated with cytotoxicity and host immune responses, thus limiting potential studies in animals and in humans (1,2). Virus infection requires successful and efficient binding of viral particles to the plasma membrane and their entry into the cell (3). This binding is thought to be mediated by specific interactions between envelope proteins and host cell surface receptors. Although, it has been proposed that receptor independent binding of the virus to the plasma membrane plays an important role in helping the viral particles to reach their specific receptors prior to cell entry (4,5) no efficient method for this approach has been developed.

Small polybasic peptides derived from the transduction domains of certain proteins, such as the third α-helix of the Antennapedia (AP) homeodomain or an 11-amino acid motif from the HIV Tat protein have been shown to cross the cell membrane through a receptor independent, non-endocytic mechanism (6-8). Such cell permeable molecules have been used as “Trojan horses” to introduce biologically active hydrophilic cargo molecules such as DNA, peptides, or proteins into cells (9). Recent examples using Tat presented on the surface of bacteriophage λ or synthesized in tandem with the proteins β-galactosidase or β-glucuronidase support the enhanced uptake and delivery of the Tat-based cargo molecules (12,13).

There is a long felt need in the art for the development of new methods and compounds for facilitating the fusion of viruses with cells and for facilitating virus mediated transduction of genes or nucleic acid delivery into cells. The present invention satisfies these needs.

### SUMMARY OF THE INVENTION

The invention relates to compositions and methods for improving virus uptake into cells and tissues and for transducing nucleic acids into cells.

In one embodiment, the invention relates to a method of rendering a cell susceptible to fusion with a virus. The method comprises contacting a cell with a composition comprising a virus and a cell permeable peptide, or a fragment, modification, or derivative thereof. In one aspect of the invention, the virus and the cell permeable peptide are preincubated together before the cell is contacted with the composition. In another aspect of the invention the cell is a mammalian cell. In yet another aspect of the invention, the cell is a human cell. In a further aspect of the invention, the cell is an endothelial cell. In yet a further aspect of the invention, the cell is a skeletal muscle cell.

In another aspect of the invention, the virus is selected from the group consisting of an adenovirus, a lentivirus, an adeno-associated virus vector, a retrovirus, and a non-replicative virus.

In one embodiment of the invention, the cell permeable peptide sequence is selected from the group consisting of a polyguanidylated peptoid (N-arg 5,7,9 peptoids), a highly charged positive peptide, an antennapedia peptide, a human immunodeficiency virus (HIV) Tat peptide, and SEQ ID NOs: 1-24. The invention also relates to a nucleic acid encoding such sequences.

The invention additionally relates to a method of facilitating transduction of a nucleic acid sequence into a cell. The method comprises contacting a cell with a composition comprising a virus and a cell permeable peptide, wherein the virus comprises the nucleic acid sequence. In one aspect of the invention, the nucleic acid sequence encodes a growth factor. In yet another aspect of the invention, the growth factor is vascular endothelial growth factor (VEGF).

The invention also relates to a method of identifying a peptide, or a fragment, modification, or derivative thereof, capable of rendering a cell susceptible to fusion with a desired virus. The method comprises contacting a cell with a composition comprising a virus and a test peptide, or a fragment, modification or derivative thereof, comparing the level of fusion of the cell with the virus with the level of fusion of an otherwise identical cell and an otherwise identical virus in a composition not comprising the test peptide, or a fragment, modification or derivative thereof. A higher level of fusion of the cell contacted with the composition comprising a virus and a test peptide, or a fragment, modification or derivative thereof, compared to an otherwise identical cell contacted with an otherwise identical virus in a composition not containing the test peptide, or a fragment, modification or derivative thereof, is an indication that the test peptide, or a fragment, modification, or derivative thereof, renders the cell susceptible to fusion with the virus. In one aspect, the invention includes a peptide identified by the method.

In addition, the invention relates to a method of treating a disease or disorder mediated by overexpression of a nucleic acid sequence or a protein or peptide encoded by such a sequence. The method comprises administering to a cell or an animal overexpressing a nucleic acid sequence a composition comprising a cell permeable peptide and a virus comprising a nucleic acid sequence which is in an antisense orientation and is complementary to the nucleic acid sequence overexpressed in a disease associated with overexpression of the nucleic acid sequence. In one aspect of the invention, the cell or animal is mammalian. In yet another aspect of the invention, the cell or animal is human.

In another aspect, the invention relates to a method of treating a disease or disorder mediated by underexpression of a nucleic acid sequence or a protein or peptide encoded by such a sequence. The method comprises administering to a cell or an animal underexpressing a nucleic acid sequence a composition comprising a cell permeable peptide and a virus comprising the nucleic acid sequence which is underexpressed in the disease. In one aspect of the invention, the cell or animal is mammalian. In yet another aspect of the invention, the cell or animal is human.

The invention also relates to a kit for administering to a cell a composition comprising a desired virus and a cell permeable peptide, or a fragment, modification or derivative thereof, wherein the peptide is capable of rendering the cell susceptible to fusion with the virus. In one aspect of the invention, the cell is a mammalian cell. In another aspect of the invention, the cell is a human cell. In a further aspect of the invention, the peptide is selected from the group consisting of a highly charged positive peptide, an antennapedia peptide, an HIV Tat peptide, and a polyguanidylated peptoid. In yet a further aspect of the invention, the peptide is selected from the group consisting of SEQ ID NOs: 1-24.

Additionally, the invention relates to a method of enhancing the ability of a virus to fuse with an animal cell. The method comprises contacting a virus with a cell permeable peptide, wherein contacting the virus with the peptide enhances the ability of the virus to fuse with an animal cell.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1, comprising FIGS. 1A to 1D, depicts an analysis of how an Antennapedia derived peptide improves adenoviral infection of cells. FIG. 1A depicts images of Western blots showing expression of (green fluorescent protein) GFP and hsp90 (loading control) in COS-7 cells infected with a GFP adenoviral expression vector (Ad-GFP) pre-complexed with increasing concentrations of the antennapedia peptide (AP). FIG. 1B depicts images based on fluorescence (left) and phase contrast (right) microscopy of COS-7 cells infected with Ad-GFP in the absence (top panels) or in the presence of AP (lower panels). FIG. 1C depicts a flow cytometry profile of GFP fluorescence of control, Ad-GFP alone, and Ad-GFP pre-complexed with AP of COS-7 cells. The tabular portion of FIG. 1C shows the number of events (total and % of total events) for all conditions under the M1 and M2 markers. FIG. 1D depicts images of Western blots (lower panels) and densitometric ratio (upper panel) of GFP and hsp90 expression levels of COS7 cells infected with increasing m.o.i. of Ad-GFP in the presence of a fixed concentration of AP. The densitometric ratio shows the average expression levels of 4 different experiments expressed as mean ±s.e.m.

FIG. 2, comprising FIGS. 2A to 2C, demonstrates that antennapedia and HIV Tat peptides are efficacious at enhancing adenovirus and retrovirus uptake by cells. FIG. 2A comprises a Western blot analysis showing the effects of both AP and Tat peptides on Ad-GFP virus infection. Ad-GFP adenovirus was pre-complexed with either AP or the Tat peptide (amino acid sequence of both peptides are shown in the inset) and COS-7 cells were infected with the Ad-GFP virus in absence or in presence of either AP or Tat. GFP and hsp90 (loading control) expression were monitored by western blotting. FIG. 2B demonstrates that AP potentiates adenovirus infection in bovine aortic endothelial cells (BAEC). Western blots showing GFP and hsp90expression levels of BAEC infected either with Ad-GFP or Ad-GFP pre-complexed with AP. FIG. 2C demonstrates results from experiments in which supernatant from phoenix packaging cells expressing GFP retrovirus (Ret-GFP), in incubated with or without AP, was used to infect human umbilical vascular endothelial cells (HUVEC). Western blots (upper panel) show GFP expression levels following infection and lower panels shows fluorescence and phase contrast microscopic images of HUVEC expressing GFP.

FIG. 3, comprising FIGS. 3A to 3C demonstrates that Antennapedia peptide facilitates viral delivery in tissues and in vivo in mice. FIG. 3A demonstrates the results of en face fluorescence of a mouse carotid artery infected luminally with Ad-GFP ex vivo. Mouse carotid arteries treated with AP (left panel), infected with Ad-GFP (middle panel) or with the AP/Ad-GFP complex (right panel) were cut open and the endothelial surface imaged by fluorescence microscopy. FIG. 3B depicts enhancement of -galactosidase activity in carotid arteries infected with Ad- -gal pre-complexed with AP. The upper panel shows cross sections of carotid arteries infected Ad- -ga l (right panel) or the AP/Ad- -gal complex (left panel) stained with X-Gal. Lower panel shows -galactosidase activity in lysates from the above vessels. FIG. 3C depicts -galactosidase activity of mice adductor muscle injected with Ad- -ga l alone or pre-complexed with AP. The upper panel shows pictures of the adductor muscle infected Ad- -gal (right panel) or the AP/Ad- - gal complex (left panel) stained with X-Gal. The lower panel shows -galactosidase activity in lysates from the above muscles. Shown are mean ±s.e.m. of at least 4 tissues. *P<0.05 vs. virus alone.

FIG. 4, comprising FIGS. 4A to 4D, shows that Antennapedia peptide improves the delivery of functionally relevant genes. FIG. 4A depicts the improvement of acetylcholine (Ach)-induced relaxation of carotid arteries from eNOS-/- mice. Isolated mouse carotid arteries from eNOS -/- mice were infected luminally with an adenovirus coding for eNOS (Ad-eNOS). In vitro, Ach-dependent relaxation was monitored in either controls carotid arteries from eNOS -/- animals (white circles), infected with Ad-eNOS alone (white squares) or infected with Ad-eNOS pre-complexed with AP (black squares). FIG. 4B demonstrates increased nitric oxide release from bovine aortic endothelial cells (BAEC) infected with Ad-eNOS. BAEC were infected with either Ad-GFP or Ad-eNOS in absence (black bars) or in presence (white bars) of AP. Nitric oxide released in the tissue culture media measured as nitrite was monitored by specific chemiluminescence. This experiment was repeated 3 times with similar results. FIG. 4C shows that AP complexed with Ad-VEGF increases vascular leakage in mouse ears. Mice were injected intradermally with either a control adenovirus (Ad- -ga l; right ear) or Ad-VEGF (left ear) in absence (top left panel) or in presence (top right panel) of AP for 4 days. Vascular leakage was monitored by Evans blue extravasation (top panels) and quantified by spectrophotometry (bottom panel). FIG. 4D shows that there is increased angiogenesis following intramuscular injection of Ad-VEGF in the presence of AP in mice subjected to hindlimb ischemia. Blood vessels of the lower limbs of mice, injected with saline, AP alone, Ad-VEGF (top left panel) or Ad-VEGF pre-complexed with AP (top right panel), were stained using anti-PECAM antibody. The percentage of the PECAM positive area was evaluated from lower limb cross-sections and analyzed using image analysis software (lower panel). Shown are mean ±s.e.m. of at least 4 sections from 6 different animals. *P<0.05 vs. Ad-VEGF alone.

### DETAILED DESCRIPTION OF THE INVENTION

The invention relates generally to compositions and methods for improving virus uptake into cells and tissues and for transducing nucleic acids into cells. The invention relates more specifically to compositions and methods for the use of cell permeable peptides to render cells susceptible to entry by viruses, which in turn improves expression of transduced nucleic acids at reduced titers of virus and increases the efficacy of therapeutically relevant nucleic acids in vivo. The invention also relates to compositions and methods for incorporating peptides into viral coats.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, “alleviating a disease or disorder symptom” means reducing the severity of the symptom.

As used herein, “amino acids” are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

“Cell permeable peptide” refers to a small peptide, including a peptide derived from the transduction domain of a certain protein, such as, but without being limited to, the third α-helix of the Antennapedia (AP) homeodomain, or an 11-amino acid motif from the HIV Tat, which have been shown to cross the cell membrane through a receptor independent, non-endocytic mechanism.

A “coding region” of a gene or a nucleic acid consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, the terms “conservative variation” or “conservative substitution” refer to the replacement of an amino acid residue by another, biologically similar residue. Conservative variations or substitutions are not likely to significantly change the shape of the peptide chain. Examples of conservative variations, or substitutions, include the replacement of one hydrophobic residue such as isoleucine, valine, leucine or alanine for another, or the substitution of one charged amino acid for another, such as the substitution of arginine for lysine, glutamic for aspartic acid, or glutamine for asparagine, and the like. In addition, “conservative nucleotide substitutions” include nucleotide substitutions which do not cause the substitution of a particular amino acid for another, as most amino acids have more than one codon (see King and Stansfield (Editors), A Dictionary of Genetics, Oxford University Press, 1997). Conservative nucleotide substitutions therefore also include silent mutations and differential codon usage.

A “^{t}control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell. A “test” cell is a cell being examined.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment”, as applied to a nucleic acid, can ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

“Fusion” is used interchangeably with “infection” and refers to the process by which a virus interacts with a cell in order to transduce nucleic acids into the cell.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty =3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

“Incubation” or “preincubation” refers to mixing the components of a composition together, such as incubating a peptide and a virus together. Incubation may occur for a set amount of time prior to adding the composition to a cell, a tissue, a sample, or a subject.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound invention or be shipped together with a container which contains the identified compound. Alternatively, the instructional material can be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

An “isolated peptide” or “substantially purified peptide”, as used herein, refers to a substantially pure protein, obtained as described herein or by methods known to those of skill in the art, which may be isolated or purified by following known procedures for protein purification, wherein an assay such as an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As used herein, a “non-replicative virus” is one which cannot replicate autonomously.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “peptide” typically refers to short polypeptides.

As used herein, the term “pharmaceutically-acceptable carrier” means a chemical composition with which an appropriate peptide or derivative can be combined and which, following the combination, can be used to administer the appropriate fusion peptide to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

The term “precomplexing” is used synonymously with “incubating” or “preincubating”, as defined above.

A “prophylactic” treatment is a treatment administered to a subject who does not exhibit signs of a disease or exhibits only early signs of the disease for the purpose of decreasing the risk of developing pathology associated with the disease.

The term “protein” typically refers to large polypeptides.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl- -galactoside to the medium (Gerhardt et al., eds., 1994, Methods for General and Molecular Bacteriology, American Society for Microbiology, Washington, D.C., p. 574).

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

A “subject” of diagnosis or treatment is a mammal, including a human. Non-human animals subject to diagnosis or treatment include, for example, livestock and pets.

The term “susceptible to fusion”, as used herein, refers to the ease with which a cell fuses with a virus. “Synthetic peptides or polypeptides” mean a non-naturally occurring peptide or polypeptide. Synthetic peptides or polypeptides can be synthesized, for example, using an automated polypeptide synthesizer. Those of skill in the art know of various solid phase peptide synthesis methods.

A “therapeutic” treatment is a treatment administered to a subject who exhibits signs of pathology, for the purpose of diminishing or eliminating those signs.

A “therapeutically effective amount” of a compound is that amount of compound which is sufficient to provide a beneficial effect to the subject to which the compound is administered.

As used herein, the term “transmembrane domain” refersio the domain of a peptide, polypeptide or protein which is capable of spanning the plasma membrane of a cell.

The term “treat,” as used herein, means reducing the frequency with which symptoms are experienced by a patient or subject or administering an agent or compound to reduce the frequency with which symptoms are experienced. As used herein, “treating a disease or disorder” means reducing the frequency with which a symptom of the disease or disorder is experienced by a patient. Disease and disorder are used interchangeably herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Methods and Compositions for Making Cells Susceptible to Fusion With a Virus

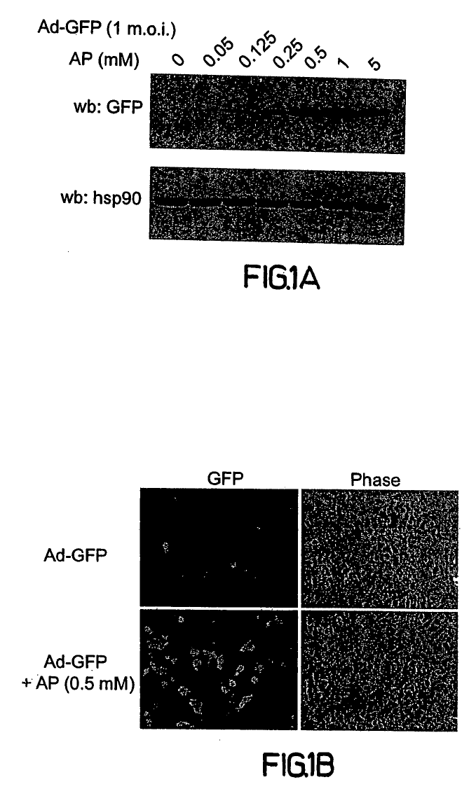

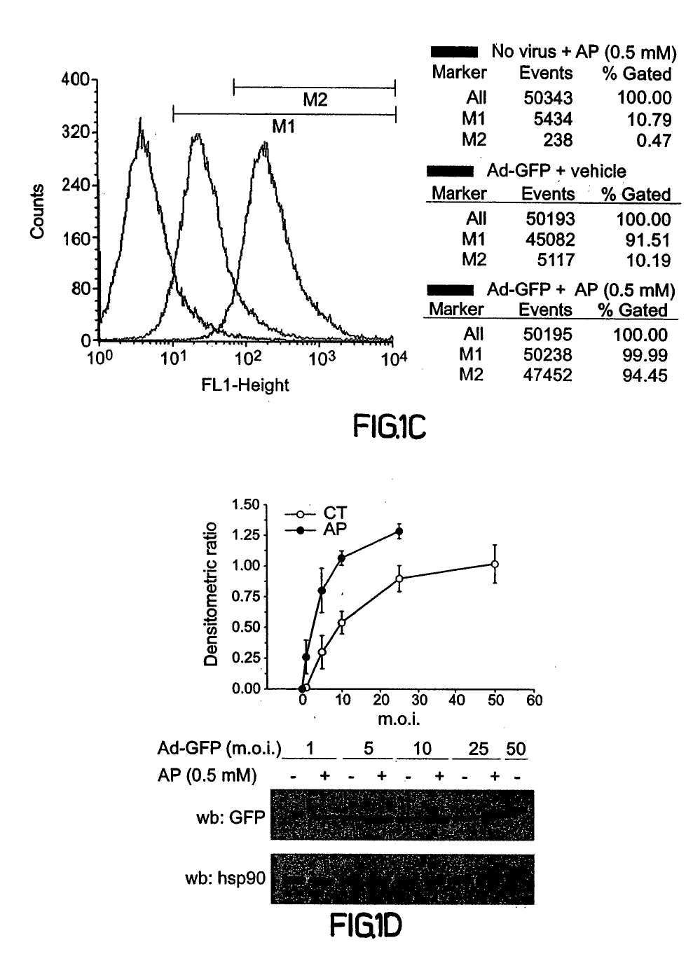

The invention, as disclosed herein, includes methods for rendering mammalian cells and viruses more capable of fusing with one another and for facilitating translocation of a nucleic acid into a cell (see FIGS. 1-4). The invention discloses a method comprising contacting a cell with a composition comprising a virus and an isolated cell permeable peptide, wherein the presence of the cell permeable peptide renders the cell and virus capable of fusing with one another.

In one embodiment of the invention, the virus and the cell permeable peptide of the composition are allowed to incubate together prior to contacting the mammalian cell with the composition. Interaction of the virus and the peptide should be construed to include electrostatic complexation of the virus wit the peptide.

In another embodiment of the invention, a nucleic acid sequence of a cell permeable peptide can be fused into the genome of the virus to be presented by the viral coat proteins to improve viral uptake.

In one aspect of the invention, the mammalian cell is an endothelial cell. In another aspect of the invention, the cell is a skeletal muscle cell. The invention should not be construed to include only skeletal muscle cells or endothelial cells, but should be construed to include other cell types as well. Preferably the cell is a mammalian cell. More preferably the mammalian cell is a human cell.

In another embodiment of the invention, the virus which is useful includes, but is not limited to, an adenovirus and a retrovirus (see FIGS. 1-4). In one aspect of the invention the retrovirus is human immunodeficiency virus (HIV). In yet another aspect of the invention, the virus may be a lentivirus, an adeno-associated viral (AAV) vector, or a non-replicative virus. One of skill in the art would recognize that other viruses would be useful in the present invention as well.

In one embodiment of the invention, a composition comprising a virus and a cell permeable peptide may be administered to a cell in vitro. In another embodiment of the invention, a composition comprising a virus and a cell permeable peptide may be administered to a cell in vivo.

It will be recognized by one of skill in the art that the virus of choice in most instances will be a replication deficient virus.

The cell permeable peptides of the present invention include highly charged positive peptides. The peptides of the invention thus include, but are not limited to, antennapedia peptide and HIV Tat peptide. In one aspect of the invention, the antennapedia peptide comprises the amino acid sequence of SEQ ID NO:1 (RQIKIWFQNRRMKWKK). In another aspect of the invention, the HIV Tat peptide comprises the amino acid sequence of SEQ ID NO:2 (GRKKRRQRRRPPQ).

In yet another aspect of the invention, a cell permeable peptide of the invention may be FGF (signal peptide) (SEQ ID NO:3; AAVLLPAVLLALLAP), Arg/Trp analogue (SEQ ID NO:4; RRWRRWWRRWWRRWRR), oligomeric D-arginine (SEQ ID NO:5; (R)₉), polyguanidylated peptoids (N-arg 5,7,9 peptoids), HSV-1 VP22 (SEQ ID NO: 6; DAATATRGRSAASRPTERPRAPARSASAPAAPVG; Schwarze and Doody, 2000, Trends Pharmacol. Sci. 21:45-48), D-Tat (SEQ ID NO:7; GRKKRRQRRRPPQ), R9-Tat (SEQ ID NO:8; GRRRRRRRRRPPQ), U2AF (142-153) (SEQ ID NO:9; SQMTRQARRLYV), HIV-1 Rev (34-50) (SEQ ID NO: 10; TRQARRNRRWRERRQR), FHV Gacoat (3549) (SEQ ID NO:11; RRRRNRTRRNRRRVR), BMV Gag (7-25) (SEQ ID NO:12; KMTRAQRRAAARIITR), HTLV-II Rex (4-16) (SEQ ID NO:13; TRRQRTRRARRNR), CCMV Gag (7-25) (SEQ ID NO:14; KLTRAQRRAAARKIIINTR), P22 N (14-30) (SEQ ID NO:15; NAKTRRHERRRKLAIER), cFos (139-164) (SEQ ID NO:16; KRRIRRERNKMAAAKSRNRRRELTDDT), cJun (252-279) (SEQ ID NO: 17; RIKAERKRMRNRIAASKSRKRKLERIAR), GCN4 (231-252) (SEQ ID NO:18; KRARNTEAARRSRARKLQRMQK), PTD-4 (SEQ ID NO: 19; PIRRRKKLRRLK), PTD-5 (SEQ ID NO:20; RRQRRTSKLMKR), Penetratin (SEQ ID NO:21; GRKKRRQRRRPPQ), Transportan (SEQ ID NO:22; GWTLNSAGYLLKINLKALAALAALIL), Amphipathic peptide (SEQ ID NO:23; KLALKLALKALKAALKLA), and HIV-1 Tat (47-58) (SEQ ID NO:24; YGRKKRRQRRR) (see Futaki et al., 2001, J. Biol. Chem. 276:8:5836-5840 for SEQ ID NOs:7-17; Mi et al., 2000, Mol. Ther. 2:339-347 for SEQ ID NOs:18-20; Lindgren et al., 2000 Trends Pharmacol. Sci. 21:99-103 for SEQ ID NOs:21 -23; and Fawell et al., 1994, Proc. Natl. Acad. Sci. USA 91:664-668 for SEQ ID NO:24).

The present invention also provides for analogs of proteins or peptides. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Pentide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the -amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxcarbonyl as the -amino protecting group, and the FMOC method which utilizes 9-fluorenylmethyloxcarbonyl to protect the -amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifing and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C8- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, ftumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in flirther processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

### Methods of Facilitating Transduction of a Nucleic Acid Sequence into a Cell

The invention disclosed herein encompasses a method of facilitating transduction of a nucleic acid sequence into a cell. In one embodiment, a virus comprising the nucleic acid sequence is preincubated or precomplexed with a cell permeable peptide useful for enhancing virus and cell fusion. Then the cell is contacted with a composition comprising the preincubated virus and cell permeable peptide. Therefore, transduction of the nucleic acid sequence is enhanced. Methods for incorporating a nucleic acid sequence into a virus are commonly used and are known-to those of skill in the art. The invention should be construed to include the various methods available for incorporating a nucleic acid sequence into a virus.

### Methods of Identifying Peptides Capable of Rendering Cells Susceptible to Fusion with a Virus

The present invention also discloses methods for identifying peptides, or fragments, modifications, or derivatives thereof, which are capable of increasing the ability of a virus and a cell to fuse with one another. The method comprises contacting a cell with a composition comprising a virus and a test peptide, or a fragment, modification, or derivative thereof, and then comparing the level of fusion of the cell with the virus with the level of fusion of a cell and a virus wherein no test peptide is present.

Methods for measuring fusion of a cell with a virus are either described herein or are known to those of skill in the art. For example, the method may include indirect measurement of fusion, such as measuring expression of a transduced nucleic acid sequence of interest, i.e., a reporter gene, wherein the virus contains the nucleic acid sequence of interest. These methods include various in vivo and in vitro assays, and various biochemical, molecular, cellular, and animal techniques.

In one embodiment of the invention, a composition comprising a virus and a cell permeable peptide may be administered to a cell in vitro. In another embodiment of the invention, a composition comprising a virus and a cell permeable peptide may be administered to a cell in vivo.

The invention includes isolated peptides or fragments, modification, or derivatives thereof, identified using the methods disclosed herein. The invention also includes an isolated nucleic acid encoding a peptide discovered by the methods described herein.

### Methods of Treating a Disease or Disorder

The invention also relates to treating diseases or disorders mediated by aberrant expression of a nucleic acid sequence. Aberrant expression should be construed to include underexpression or overexpression of a gene or nucleic acid sequence. The disclosure provides herein methods for treating diseases and disorders which include, but are not limited to, heart and vascular diseases, cancer, lung diseases, hematological disorders, neurological diseases, and diseases associated with inflammation, including arthritis, inflammatory bowel disease and Crohn's disease.

In one embodiment, a composition comprising a cell permeable peptide and a virus comprising a nucleic acid sequence is administered to a cell which is underexpressing the nucleic acid sequence or is administered to an animal underexpressing the nucleic acid sequence. Underexpression of the nucleic acid sequence is associated with a disease or disorder in the animal.

In another embodiment of the invention, a composition comprising a cell permeable peptide and a virus comprising a nucleic acid sequence which is in an antisense orientation and is complementary to a nucleic acid sequence is administered to a cell overexpressing the nucleic acid sequence or is administered to an animal overexpressing the nucleic acid sequence. Overexpression of the nucleic acid sequence is associated with a disease or disorder in the animal.

In one embodiment of the invention the nucleic acid sequence of interest encodes a growth factor. In one aspect of the invention, the growth factor is vascular endothelial growth factor.

It will be appreciated by one of skill in the art that when a gene or nucleic acid is underexpressed, that replacement of expression of the gene or nucleic acid need not necessarily be in a cell which is underexpressing the gene or nucleic acid.

It will be recognized by one of skill in the art that the virus of choice in most instances will be a replication deficient virus.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

It will be recognized by one of skill in the art that the peptides, molecules, viruses, and compositions of the invention can be administered in vitro to cells or tissues as part of an ex vivo therapy or use for cells or tissues which will then be returned to the subject.

The invention relates to the administration of an identified peptide and a virus in a pharmaceutical composition to practice the methods of the invention, the composition comprising the peptide or an appropriate derivative, modification, or fragment of the peptide, a virus, and a pharmaceutically-acceptable carrier.

In one embodiment, the pharmaceutical compositions useful for practicing the invention may be administered to deliver a dose of between 1 ng/kg/day and 100 mg/kg/day.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parerterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer, for example, peptides, fragments, modifications or derivatives thereof, and a virus comprising a nucleic acid sequence of interest according to the methods of the invention. The method should not be construed to be limited to the peptides described herein, but should be construed to include other peptides, fragments or derivatives thereof.

Peptides which are identified using any of the methods described herein may be formulated and administered to a mammal for treatment of various diseases described herein.

The invention encompasses the preparation and use of pharmaceutical compositions comprising a cell permeable peptide and a virus for diseases described herein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffm, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may fturther comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i. e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (iLe., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may furter comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infuasion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may farther comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The phannaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifingal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

### Kits for Administering Peptides and Viruses

The method of the invention includes a kit comprising a cell permeable peptide identified in the invention, a virus, and an instructional material which describes administering the composition to a cell or an animal. It will be recognized by one of skill in the art that the virus of choice in most instances will be a replication deficient virus. The invention should be construed to include other embodiments of kits that are known to those skilled in the art, such as a kit comprising a (preferably sterile) solvent suitable for dissolving or suspending the composition of the invention prior to administering the compound to a cell or an animal. Preferably the animal is a human.

### Biochemical, Molecular Biology, Microbiology and Recombinant DNA Techniques

In accordance with the present invention, as described above or as discussed in the Examples below, there can be employed conventional biochemical, molecular biology, microbiology and recombinant DNA techniques which are known to those of skill in the art. Such techniques are explained fully in the literature. See for example, Sambrook et al., 1989 Molecular Cloning—a Laboratory Manual, Cold Spring Harbor Press; Glover, (1985) DNA Cloning: a Practical Approach; Gait, (1984) Oligonucleotide Synthesis; Harlow et al., 1988 Antibodies—a Laboratory Manual, Cold Spring Harbor Press; Roe et al., 1996 DNA Isolation and Sequencing: Essential Techniques, John Wiley; and Ausubel et al., 1995 Current Protocols in Molecular Biology, Greene Publishing.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

The materials and methods used in the present invention are now described.

Peptides and Viruses

Peptides, corresponding to the antennapedia internalization sequence (amino acids 43-58: RQIKIWFQNRRMKWKK; SEQ ID NO:1) or the HIV Tat (amino acids 48-60: GRKKRRQRRRPPQ; SEQ ID NO:2) were synthesized by standard Fmoc chemistry and analyzed by reversed-phase HPLC and mass spectrometry by the W.M. Keck biotechnology resource center at Yale University School of Medicine. Peptides were dissolved in deionized water (stock solution: 25 mM) and sterile filtered before use.

Replication deficient adenovirus coding for GFP, -galactosidase, endothelial nitric oxide synthase and VEGF were generated and amplified as described previously (11,14). Replication deficient retrovirus coding for GFP was produced as described previously using the phoenix packaging cell line (15). Supernatant from these cells was used directly for infections.

Animals

C57BL/6J (Charles River Laboratory, Wilmington, Mass.), eNOS knock out (NOS3, Jackson Laboratory, Bar Harbor, Me.) or CD1 (Charles River Laboratory, Wilmington, Mass.) 8-12 week-old, male mice were used. All surgical procedures were done under ketamine/xylazine anesthesia (79.5 mg/kg ketamine, 9.1 mg/kg xylazine). The Institutional Animal Care and Use Committee of Yale University approved all procedures.

Cell Infection

Adenoviruses (1 m.o.i. or approximately 1×10⁶ pfu per 60 mm dish) were pre-complexed with the peptides (0.05 to 5 mM) in 100 μl of serum free media (OptiMEM; Gibco, Grand Island, N.Y.) for 30 min at room temperature. Cells (COS-7 or BAEC) were grown on 60 mm dishes until 90% confluent and media was changed to serum free (2 ml) before exposure to the adenovirus complex. The complex (100 μl) was added to the cells for 4 hrs and then the cells were washed and changed to complete growth media for 24 to 48 hours.

For retroviral infections supernatant from phoenix retroviral packaging cell line expressing Ad-GFP was incubated for 30 min at 37 C in presence of AP (0.1-0.5 mM). HUVEC cells were exposed to the mixture for 24 hrs and then harvested.

Western Blotting

Western blotting was performed as described previously (16). Briefly, cells were lysed (50 mM Tris-HCl, 0.1 mM EDTA, 0.1 mM EGTA, 1% (v/v) Nonidet P-40, 0.1% SDS, 0.1% deoxycholic acid, 1 mM Pefabloc, 10 μg/ml aprotinin, and 10 μg/ml leupeptin) for 1 hr at 4° C. and the soluble material was separated on a 10% SDS-PAGE, and transferred to a nitrocellulose membrane. Blocked membranes were probed using the specific polyclonal anti-GFP (Clontech, Palo Alto, Calif.) or the monoclonal anti-hsp90(Stressgen, Victoria, Canada) antibodies. After washing and incubating with secondary antibodies, immunoreactive proteins were visualized by ECL detection system (Amersham, Buckinghamshire, UK).

Fluorescent Microscopy and Flow Cyometry

Live cells infected with Ad-GFP were washed with serum free media and visualized using a Zeiss Axiovert 200 microscope under phase contrast or GFP filters (excitation 450 nm, emission 510 nm). Images were captured using the Openlab Imaging software (Improvision, Lexington, Mass.). Flow cytometric analysis of GFP expressing cells was performed 48 h after following infection. Cells were trypsinized, PBS washed and fixed in 70% ethanol at 4° C. Cells were resuspended in PBS and were analyzed by gating for GFP fluorescence by flow cytometry using a FACSort (Becton Dickinson, San Jose, Calif.).

Nitric Oxide Release

Basal NO2⁻ release from BAEC, a stable metabolite of NO, was assessed in the medium as described (9). In all experiments, release was attenuated by a NOS inhibitor.

Endothelium Specific Gene Transfer of Mouse Carotid Arteries

Mice were anesthetized and exsanguinated via the inferior vena cava followed by perfusion of saline through the left ventricle. The common carotid artery was cannulated, flushed with a small amount (˜3 μl) of virus and tied off proximally. Approximately 3 μl of Ad-β-gal (1×10⁹ pfu/ml), Ad-GFP (1×10⁹ pfu/ml), Ad-eNOS (5×10¹⁰ pfu/ml) virus or virus complexed with AP (10 μM) for 30 min at room temperature was injected. The virus-filled vessel was incubated in situ at 37° C. for 2 hours and then rinsed in saline prior to overnight (18 hours) incubation in complete DMEM at 37° C., 5% CO₂. Control vessels were filled with viral storage buffer and treated in an identical manner.

Isometric Studies

Rings of carotid artery were studied as described previously (17). To assess endothelial function, vessels were precontracted with a submaximal (˜80%) concentration of PGF_{2α} (5×10⁻⁶ mol/L) prior to application of endothelium-dependent dilator acetylcholine (10⁻¹⁰−3×10⁻⁶ mol/L).

-galactosidase Gene Expression

-galactosidase expression was monitored by either X-Gal staining or activity 4 days after virus administration as described previously (14,18).

Permeability Assay

Ad-VEGF (5.5×10⁷ pfu) or Ad-β-gal (2.5×10⁷ pfu) with or without AP complex (10 μM; 30 minutes at room temperature) was injected intradermally into the ear of CD1 mouse. Evans blue leakage was monitored 4 days after infection as described previously (9).

Ischemic Hind Limb Angiogenesis Assay

Ischemic hind limb model was performed as described previously (19). Briefly, following anesthesia, the left femoral artery was exposed under a dissection microscope. The proximal end of the femoral artery and the distal portion of the saphenous artery were ligated. All branches between these two sites were cauterized, and arteriectomy was performed. Following arteriectomy, 25 μl of either virus storage buffer, Ad-VEGF (5.5×10⁷ pfu), Ad- -ga l (2.5×10⁷ pfu), pre-complexed with AP (30 min room temperature; 10 μM) or AP alone (at least 5 animals per group) was injected into the adductor muscle at 3 different sites. Mice were sacrificed 21 days following surgery and muscles of the lower limbs were harvested, methanol fixed and paraffm embedded. Tissue sections (5 μm thick) were stained using an anti-CD31 antibody (Pharmingen, San Diego, Calif.), and hematoxylin counter stained. Pictures from 4 random areas of each section, and 3 sections per mice were taken using a Kodak digital camera mounted on a light microscope (40× objective). Capillary density was quantified by measuring the percentage of CD31 positive area out of total area using the Matlab software (The Math Works, Inc.).

Statistical Analysis

Data are expressed as means ±s.e.m. Statistical differences were measured by either Student's T-test, one or two-way analysis of variance followed by Bonferonni post-hoc test P 0.05 was considered as significant.

### EXAMPLE 1

One possible mechanism to improve cell surface concentrations of viral particles may be through the use of cell permeable peptides. Using cell permeable peptides, an efficient and simple method to increase virally mediated gene delivery, and thus protein expression in cells in vitro and in vivo, has been developed and is described herein.

To test the effects of pre-complexing adenoviruses with cell permeable peptides prior to cell infection by preincubating the virus with the peptide, COS-7 cells were used as target cells to monitor virally mediated transgene expression with an adenovirus encoding green fluorescent protein (Ad-GFP). Increasing concentrations of a synthetic peptide representing amino acids 43-58 (SEQ ID NO:1) of antennapedia (AP) was used against fixed amounts of Ad-GFP (1 multiplicity of infection; m.o.i., FIG. 1A). AP (0.05 to 5.0 mM) was pre-incubated with Ad-GFP for 30 min at room temperature in serum free media (100 μl; see methods section) prior to infection, and then diluted 20 fold into tissue culture medium. As seen in FIG. 1A, pre-incubation of Ad-GFP with AP dose-dependently improved expression of GFP, as revealed by western blotting of GFP (FIG. 1A) without influencing the levels of hsp90 expression as a control for protein loading. Exposure of cells to AP prior to or after Ad-GFP infection did not yield an increase in GFP expression, thus indicating that the pre-complex step is necessary for improvement of cell infection.

Visualization of cells by fluorescent microscopy showed increased numbers of GFP positive cells when Ad-GFP is pre-incubated with 0.5 mM of AP (FIG. 1B) compared to virus alone. This was confirmed by flow cytometric analysis. As seen in FIG. 1C, Ad-GFP infection at 1 m.o.i. yielded approximately 92% of the cells expressing low level GFP (M1, green population), with fewer cells expressing high levels of GFP. In contrast, pre-complexation of Ad-GFP with AP yielded an approximate 10-fold increase in cells strongly positive for GFP (M2, red population). In order to determine to what degree AP influenced the titer necessary for robust expression, AP was complexed (at a concentration of 0.5 mM in the pre-complexing step) with increasing amounts of Ad-GFP (FIG. 1D). The densitometric ratio of GFP to hsp90 expression levels revealed that a 5 to 10 fold lower titer of Ad-GFP is sufficient for similar expression of GFP in presence of AP (FIG. 1D; upper panel). Thus, pre-incubation of adenovirus with AP improves transduction efficiency.

### EXAMPLE 2

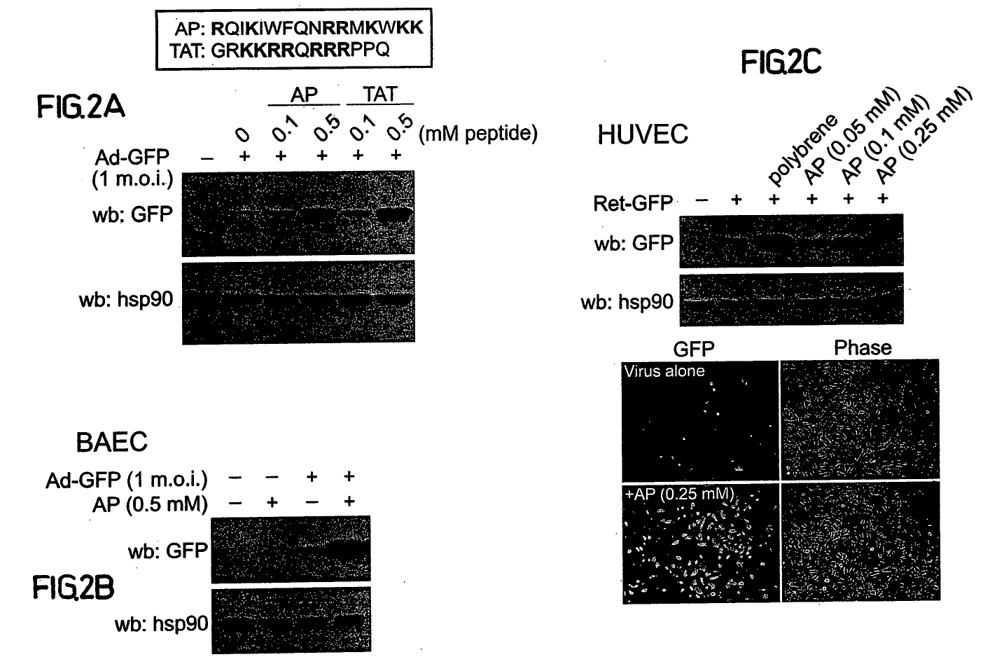

To test whether another known cell permeable peptide has similar effects on adenoviral infection, HIV derived Tat peptide (amino acids 48-60; SEQ ID NO:2) (FIG. 2A; upper panel) was used. The Tat peptide (0.1 and 0.5 mM) was shown to be as efficient as AP at increasing Ad-GFP infection in COS-7 cells (FIG. 2A; lower panel) indicating that this property may be a common feature of polybasic cell permeable peptides. Next, it was determined whether enhanced infection, in the presence of AP, is seen in a primary cell line where adenoviral mediated transgene expression is more difficult. Thus, bovine aortic endothelial cells (BAEC) were exposed to either Ad-GFP alone or the AP/Ad-GFP complex and expression of GFP was monitored by western blotting (FIG. 2B). A marked increase in GFP expression levels was observed in the presence of AP compared to Ad-GFP alone, however the levels of hsp90 remained constant (FIG. 2B). In addition, it was determined whether AP could influence the expression of retrovirally-mediated expression of GFP. The addition of AP (0.05-0.5 mM) to the viral supernatant (approximately 10⁶-10⁷ pfu/ml) of phoenix packaging cells producing a retrovirus coding for GFP (Ret-GFP), dose-dependently enhanced GFP expression in human umbilical vascular endothelial cells (HUVEC) (FIG. 2C; upper panel). The effects of AP on Ret-GFP infection were similar to the effects of polybrene, a poly-cationic compound commonly used to promote retroviral infection (10). Fluorescence microscopy showed a substantial increase in GFP positive cells in the presence of AP/Ret-GFP complex (FIG. 2C; lower panel).

### EXAMPLE 3

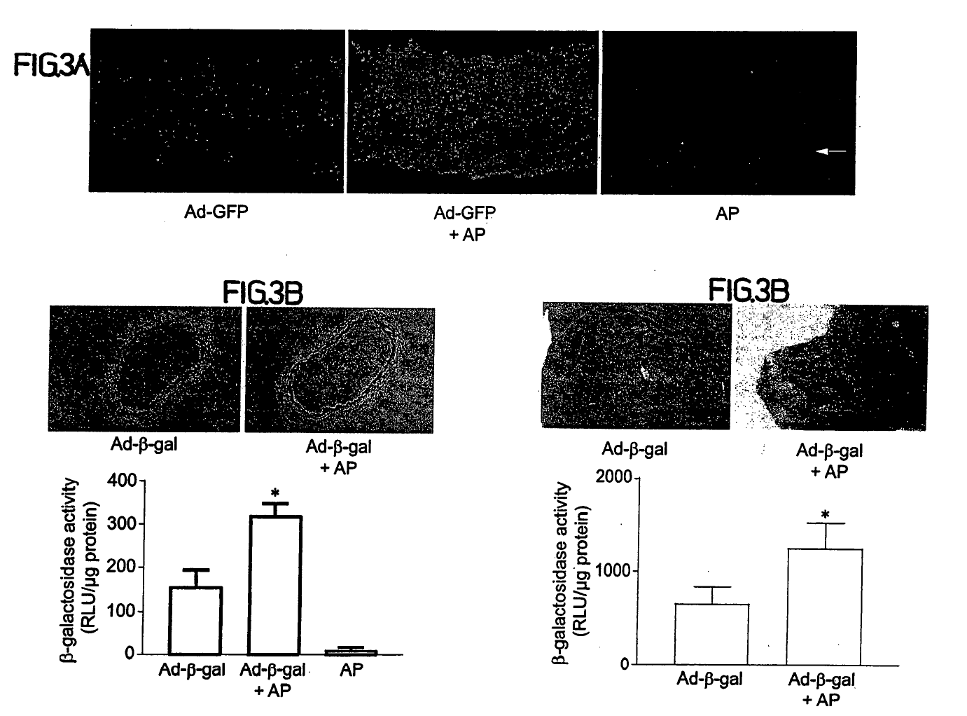

The next series of experiments were designed to validate the effects of these cell permeable peptides in a physiological setting by examining adenoviral gene transfer to tissue. First, mouse carotid arteries were infected ex vivo by luminal administration of Ad-GFP to target vascular endothelial cells. En face fluorescence imaging of the carotid artery shows that Ad-GFP (10⁹ pfu/ml) pre-complexed with AP (final concentration, 10 μM) markedly improved the infectivity of the Ad-GFP virus (FIG. 3A). Similarly, using a β-galactosidase reporter adenovirus (Ad-β-gal, 10⁹ pfu/ml), AP pre-treatment increased staining in the endothelial cell layer of the mouse carotid artery as seen in the tissue cross section (FIG. 3B; upper panel). This effect was also seen when β-galactosidase activity was measured in these vessels following infection. In carotid arteries treated with the AP/Ad-β-gal complex, β-galactosidase activity was increased by two fold over Ad-β-gal infection alone (FIG. 3B; lower panel). The effects of AP were also investigated by intramuscular administration of the Ad-β-gal reporter virus. Injection of a low titer of Ad-β-gal (5×10⁷ pfU in 25 μl, divided and injected into three sites) into the left adductor muscle of a mouse revealed low-level staining for β-galactosidase activity. This staining was increased when Ad-β-gal virus was pre-complexed with AP (final concentration, 10 μM) prior to injection in the mice (FIG. 3C; upper panel). These results were corroborated by the two-fold increase in β-galactosidase activity in the adductor muscle of mice injected with the AP/Ad-β-gal complex versus β-gal alone (FIG. 3C; lower panel). Thus, AP improves the efficiency of adenoviral uptake into the endothelium and skeletal muscle.

### EXAMPLE 4

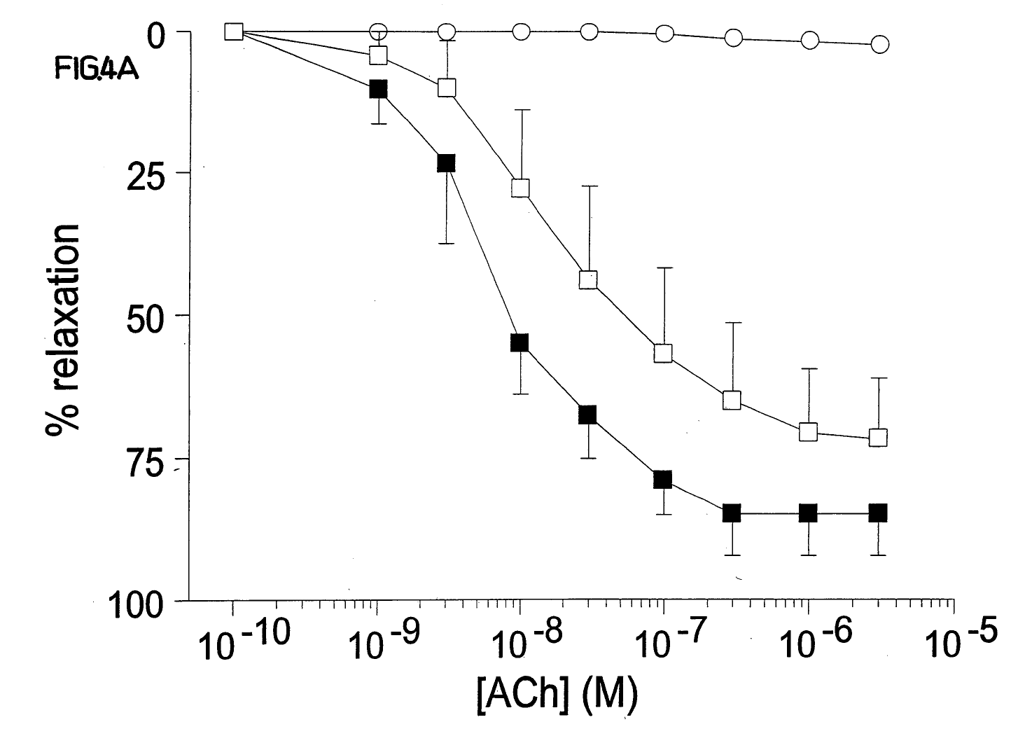

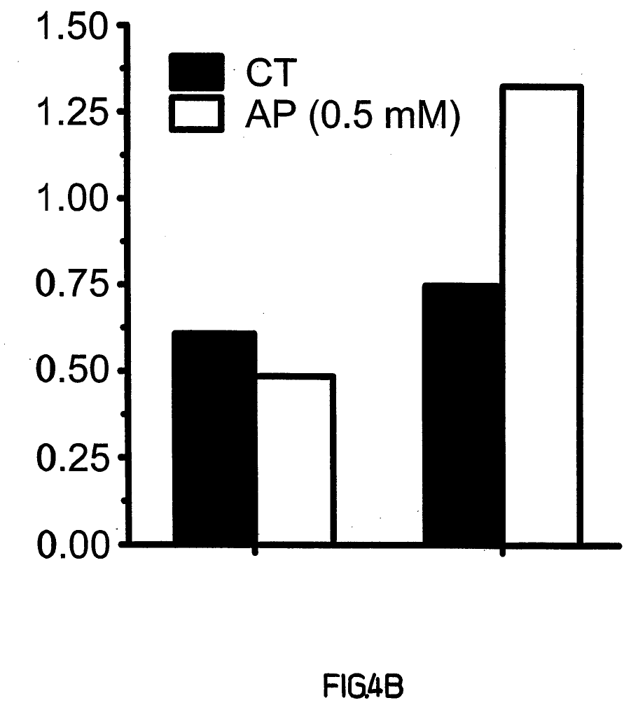

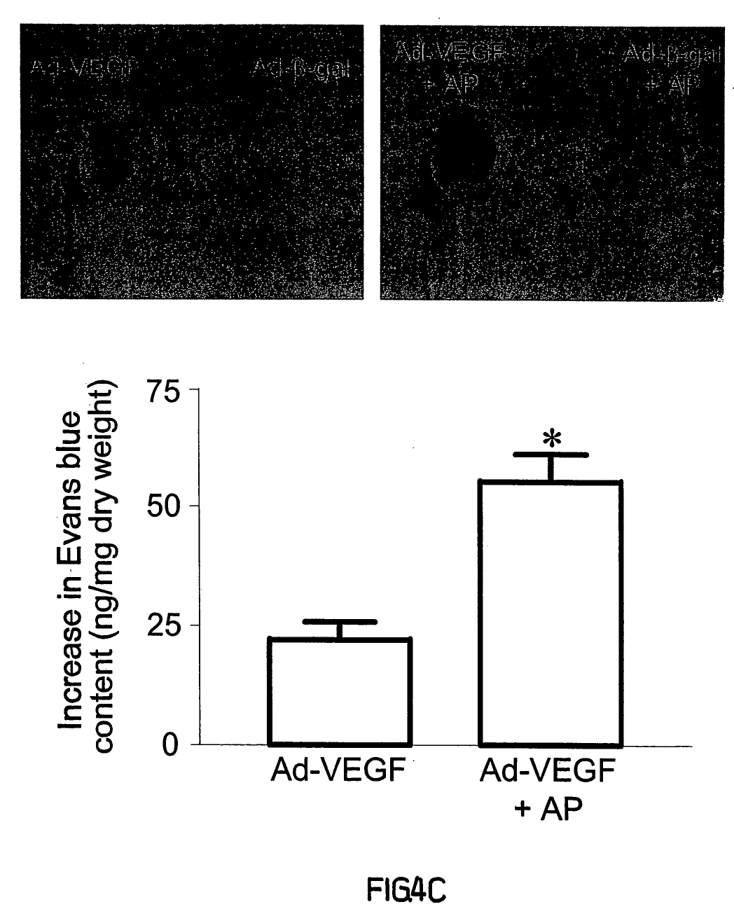

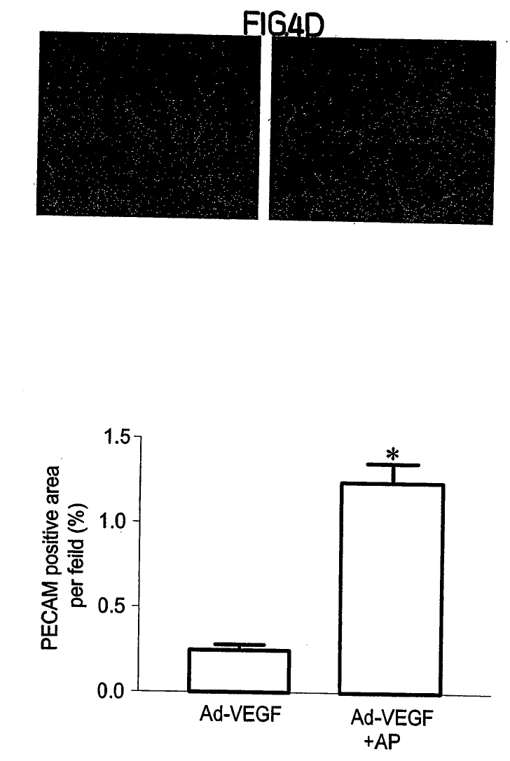

To address whether AP can improve the delivery of a functionally relevant gene, carotid arteries from endothelial nitric oxide synthase deficient mice (eNOS -/-) were luminally transduced with Ad-eNOS or Ad-eNOS pre-complexed with AP. As seen in FIG. 4A, gene transfer of Ad-eNOS to the endothelium of eNOS (-/-) mice restores endothelium dependent dilation in response to acetylcholine (Ach), an effect improved by AP. This improvement of endothelium-dependent responses is due to augmented nitric oxide (NO) production because AP improves Ad-eNOS mediated NO release from cultured endothelial cells (FIG. 4B, 1 m.o.i. of eNOS virus in the absence or presence of AP). Infection of endothelial cells with the Ad-GFP (1 m.o.i.) as a control virus, in absence or presence of AP, does not influence NO release.

Next, two angiogenic properties of adenoviral mediated VEGF (Ad-VEGF) expression in mice were monitored: vascular permeability and new blood vessel formation in the ischemic hind limb. Typically, 10⁹ pfu of Ad-VEGF will improve angiogenesis in vivo (11). Here, 5.5×10⁷ pfu were used in the absence or presence of AP. AP significantly enhanced the ability of VEGF to promote vascular leak (FIG. 4C, top panel) as indexed by the extravasation of Evans blue. AP complexed with Ad-VEGF increased basal vascular permeability by 2.5 fold over Ad-VEGF alone (FIG. 4C). Moreover, intramuscular injection of Ad-VEGF, in the presence of AP, significantly increased the angiogenic potential of the growth factor as indexed by PECAM positive vessels (>4 fold compared to Ad-VEGF alone) in the lower limbs of mice subjected hind limb ischemia. Collectively, these data demonstrate that cell permeable peptides such as AP and TAT are very efficacious at promoting the cellular entry of adenovirus and thus improve gene expression at lower titers of virus in vitro and therapeutic gene delivery in vivo.

The studies described herein provide a simple method to markedly improve viral delivery to cells and tissues using cell permeable peptides. Without wishing to be bound by theory, it is suspected that highly positive charged peptides like AP and Tat, interact with either the protein (adenovirus) or lipid coat (retrovirus) of the viruses and improve the effective surface concentration of viral particles subsequent to receptor dependent uptake mechanisms. The discoveries of the present invention, extend this theory by showing that electrostatic coupling of AP or Tat improves virus mediated gene delivery, thus raising the possibility that cell permeable peptides, complexed in solution (described herein) or perhaps fused into the viral coat, are useful adjuncts to therapeutic gene targeting.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### References

1. Chen, A. F., O'Brien, T. & Katusic, Z. S. Transfer and expression of recombinant nitric oxide synthase genes in the cardiovascular system. Trends Pharmacol Sci 19, 276-86. (1998).
2. Baek, S. & March, K. L. Gene therapy for restenosis: getting nearer the heart of the matter. Circ Res 82, 295-305. (1998).
3. Cullen, B. R. Journey to the center of the cell. Cell 105, 697-700. (2001).
4. Pizzato, M., Marlow, S. A., Blair, E. D. & Takeuchi, Y. Initial binding of murine leukemia virus particles to cells does not require specific Env-receptor interaction. J Virol 73, 8599-611. (1999).
5. Sharma, S., Miyanohara, A. & Friedmann, T. Separable mechanisms of attachment and cell uptake during retrovirus infection. J Virol 74, 10790-5. (2000).
6. Schwarze, S. R. & Dowdy, S. F. In vivo protein transduction: intracellular delivery of biologically active proteins, compounds and DNA. Trends Pharmacol Sci 21, 45-8. (2000).
7. Schwarze, S. R., Ho, A., Vocero-Akbani, A. & Dowdy, S. F. In vivo protein transduction: delivery of a biologically active protein into the mouse. Science 285, 1569-72. (1999).
8. Derossi, D., Chassaing, G. & Prochiantz, A. Trojan peptides: the penetratin system for intracellular delivery. Trends Cell Biol 8, 84-7. (1998).
9. Bucci, M. et al. In vivo delivery of the caveolin-1 scaffolding domain inhibits nitric oxide synthesis and reduces inflammation. Nat Med 6, 1362-7. (2000).
10. Le Doux, J. M., Landazuri, N., Yarmush, M. L. & Morgan, J. R. Complexation of retrovirus with cationic and anionic polymers increases the efficiency of gene transfer. Hum Gene Ther 12, 1611-21. (2001).
11. Rivard, A. et al. Rescue of diabetes-related impairment of angiogenesis by intramuscular gene therapy with adeno-VEGF. Am J Pathol 154, 355-63. (1999).
12. Eguchi, A. et al. Protein transduction domain of HIV-1 Tat protein promotes efficient delivery of DNA into mammalian cells. J Biol Chem 276, 26204-10. (2001).
13. Xia, H., Mao, Q. & Davidson, B. L. The HIV Tat protein transduction domain improves the biodistribution of beta-glucuronidase expressed from recombinant viral vectors. Nat Biotechnol 19, 640-4. (2001).
14. Luo, Z. et al. Acute modulation of endothelial Akt/PKB activity alters nitric oxide-dependent vasomotor activity in vivo. J Clin Invest 106, 493-9. (2000).
15. Zheng, L. et al. Cytoprotection of human umbilical vein endothelial cells against apoptosis and CTL-mediated lysis provided by caspase-resistant Bcl-2 without alterations in growth or activation responses. J Immunol 164, 4665-71. (2000).
16. Gratton, J. P. et al. Akt down-regulation of p38 signaling provides a novel mechanism of vascular endothelial growth factor-mediated cytoprotection in endothelial cells. J Biol Chem 276, 30359-65. (2001).
17. Rudic, R. D., Bucci, M., Fulton, D., Segal, S. S. & Sessa, W. C. Temporal events underlying arterial remodeling after chronic flow reduction in mice: correlation of structural changes with a deficit in basal nitric oxide synthesis. Circ Res 86, 1160-6. (2000).
18. Schulick, A. H., Dong, G., Newman, K. D., Virmani, R. & Dichek, D. A. Endothelium-specific in vivo gene transfer. Circ Res 77, 475-85. (1995).
19. Couffinhal, T. et al. Mouse model of angiogenesis. Am J Pathol 152, 1667-79. (1998).

1. A method of rendering a cell susceptible to fusion with a desired virus, said method comprising contacting said cell with a composition comprising said virus and an isolated cell permeable peptide, or a fragment, modification or derivative thereof, wherein said peptide, or a fragment, modification or derivative thereof, is capable of rendering said cell susceptible to fusion with said virus, thereby rendering a cell susceptible to fusion with a desired virus.

2. The method of claim 1, wherein said virus and said peptide, or a fragment, modification or derivative thereof, are preincubated together prior to contacting said cell with said coimposition.

3. The method of claim 1, wherein said virus is selected from the group consisting of an adenovirus, a lentivirus, an adeno-associated virus vector, a retrovirus, and a non-replicative virus.

4. The method of claim 1, wherein said retrovirus is human immunodeficiency virus (HIV).

5. The method of claim 1, wherein said cell is a mammalian cell.

6. The method of claim 5, wherein said mammalian cell is a human cell.

7. The method of claim 1, wherein said cell is selected from the group consisting of an endothelial cell and a skeletal muscle cell.

8. The method of claim 1, wherein said peptide, or a fragment, modification or derivative thereof, is selected from the group consisting of SEQ ID NOs:3 to 24, a polyguanidylated peptoid, a highly charged positive peptide, an antennapedia peptide, and a human immunodeficiency virus (HIV) Tat peptide.

9. The method of claim 8, wherein said antennapedia peptide comprises the amino acid sequence of SEQ ID NO:1.

10. The method of claim 8, wherein said HIV Tat peptide comprises the amino acid sequence of SEQ ID NO:2.

11. A composition comprising said composition of claim 1 and a pharmaceutically acceptable carrier.

12. An isolated nucleic acid encoding said peptide, or a fragment, modification or derivative thereof, of claim 1.

13. The method of claim 1, wherein said cell is contacted in vitro.

14. The method of claim 1, wherein said cell is contacted in vivo.

15. A method of facilitating transduction of a nucleic acid sequence into a cell, said method comprising the method of claim 1, further wherein said virus comprises said nucleic acid sequence, thereby facilitating transduction of a nucleic acid sequence into a cell.

16. A composition comprising said virus of claim 15 and a pharmaceutically acceptable carrier.

17. The method of claim 15, wherein said nucleic acid sequence encodes a growth factor.

18. The method of claim 17, wherein said growth factor is vascular endothelial growth factor (VEGF).

19. An isolated nucleic acid encoding a cell permeable peptide, wherein said cell permeable peptide renders a cell susceptible to fusion with a virus, wherein said virus comprises a nucleic acid sequence, further wherein said cell permeable peptide facilitates transduction of said nucleic acid sequence into said cell.

20. An isolated peptide encoded by the nucleic acid sequence of claim 19.

21. A method of identifying a peptide, or a fragment, modification or derivative thereof, capable of rendering a cell susceptible to fusion with a desired virus, said method comprising contacting a cell with a composition comprising said virus and a test peptide, or a fragment, modification or derivative thereof, comparing the level of fusion of said cell with said virus with the level of fusion of an otherwise identical cell and an otherwise identical virus in a composition not comprising said test peptide, or a fragment, modification or derivative thereof, wherein a higher level of said fusion in said cell contacted with said composition comprising a virus and a test peptide, or a fragment, modification or derivative thereof, compared to said otherwise identical cell contacted with said otherwise identical virus in a composition not containing said test peptide, or a fragment, modification or derivative thereof, is an indication that said test peptide, or a fragment, modification, or derivative thereof, renders said cell susceptible to fusion with said virus, thereby identifying a peptide, or a fragment, modification or derivative thereof, capable of rendering a cell susceptible to fuision with a desired virus.

22. A peptide, or a fragment, modification, or derivative thereof identified by the method of claim 21.

23. An isolated nucleic acid encoding the peptide of claim 22.

24. A method of treating a disease or disorder mediated by lack of expression or underexpression of a nucleic acid sequence, said method comprising administering to a cell lacking expression or underexpressing said nucleic acid sequence a composition comprising a virus and a cell permeable peptide capable of facilitating fusion of said virus with said cell, further wherein said virus comprises said nucleic acid sequence, wherein said nucleic acid sequence is expressed upon transduction into said cell, thereby treating a disease or disorder mediated by lack of expression or underexpression of a nucleic acid sequence.

25. A method of treating a disease or disorder mediated by overexpression of a nucleic acid sequence, said method comprising administering to a cell overexpressing said nucleic acid sequence a composition comprising a virus and a cell permeable peptide capable of facilitating fusion of said virus with said cell, further wherein said virus comprises a nucleic acid sequence encoding an isolated nucleic acid complementary to said nucleic acid sequence, or a fragment thereof, said isolated complementary nucleic acid being in an antisense orientation, said composition further comprising a pharmaceutically-acceptable carrier, thereby treating a disease or disorder mediated by overexpression of a nucleic acid.

26. A kit for administering to a cell a composition comprising a desired virus and a cell permeable peptide, or a fragment, modification or derivative thereof, wherein said peptide is capable of rendering said cell susceptible to fusion with said virus, said kit comprising said virus, said peptide, or a fragment, modification, or derivative thereof, an applicator, and an instructional material for the use thereof.

27. The kit of claim 26, wherein said cell is a mammalian cell.

28. The kit of claim 27, wherein said cell is a human cell.

29. The kit of claim 26, wherein said peptide is selected from the group consisting of a highly charged positive peptide, an antennapedia peptide, and an HIV Tat peptide.

30. The kit of claim 29, wherein said antennapedia peptide comprises the amino acid sequence of SEQ ID NO:1.

31. The kit of claim 29, wherein said HIV Tat peptide comprises the amino sequence of SEQ ID NO:2.

32. A method of enhancing the ability of a virus to fuse with an animal cell, said method comprising contacting said virus with an isolated cell permeable peptide, wherein contacting said virus with said cell permeable peptide enhances the ability of said virus to fuse with an animal cell, thereby enhancing the ability of a virus to fuse with an animal cell.
